(19) United States
(12) Patent Application Publication
(10) Pub. No.: US 2026/0345620 A1
(43) Pub. Date: Nov. 5, 2026

(54) IMPROVEMENTS IN OR RELATING TO COMPOSITIONS FOR TREATMENT OF LESIONS

(71) Applicants: Anton ALEXANDROFF, Cambridge (GB); SYDERMA LTD, London (GB)

(72) Inventor: Anton ALEXANDROFF, Cambridge (GB)

(21) Appl. No.: 19/486,806

(22) PCT Filed: May 29, 2024
(86) PCT No.: PCT/GB2024/051370

§ 371 Date: Nov. 21, 2025

(30) Foreign Application Priority Data
Jun. 3, 2023 (GB) 2308326.4

(51) Int. Cl. A61K 31/522; A61K 31/513; A61P 17/12; A61P 35/00

(52) U.S. Cl. CPC A61K 31/522; A61K 31/513; A61P 17/12; A61P 35/00

(57) ABSTRACT

Disclosed is a composition comprising 5-fluorouracil (5-FU) and a composition comprising an inhibitor of Ataxia Telangiectasia Mutated (ATM) kinase, Ataxia Telangiectasia Related (ATR) kinase and Poly-ADP-ribose polymerase (PARP) inhibitor, for treatment of a hyperproliferative keratinocyte disorder of the skin and/or mucous membrane of a mammalian, preferably human, subject; a composition comprising both 5-fluorouracil (5-FU) and an inhibitor of Ataxia Telangiectasia Mutated (ATM) kinase, Ataxia Telangiectasia Related (ATR) kinase and Poly-ADP-ribose polymerase (PARP) inhibitor, for treatment of a hyperproliferative keratinocyte disorder of the skin and/or mucous membrane of a mammalian, preferably human, subject; a method of making said composition; and a method of treating a hyperproliferative keratinocyte disorder of the skin and/or mucous membrane of a mammalian, preferably human, subject, using the composition/s of the invention.

### FIELD OF THE INVENTION

The present invention relates to a composition for treatment of lesions, to a method of making the composition, and to uses of the composition.

### BACKGROUND OF THE INVENTION

While a number of topical treatments such as 5-fluorouracil and imiquimod are currently available actinic keratoses and basal cell carcinomas, these treatments have limitations: their efficacy may be only about 50-60% for actinic keratoses and 60-70% and 70-80% for basal cell carcinomas; and/or they can cause a painful, severe inflammatory reaction in 20-50% percent of patients (refs. 1,2, 3).

Hyperplastic disorders involve some level of accelerated and also dysregulated cellular proliferation. In these cases, cells may stop reacting appropriately to external proliferation signals, continuing to proliferate even in the absence of enabling proliferation signals. Furthermore, the intrinsic proliferation-promoting signals may overcome opposing inhibitory signals, enabling cells to continue to progress through the cell cycle regardless.

One of the key proliferation-inhibitory signals that arrest otherwise proliferating cells is DNA damage and the associated signalling response. DNA damage in general, and double-strand DNA breaks in particular, need to be resolved before mammalian cells can move from the G0/1 phase of the cell cycle into the S phase, associated with DNA replication, the key step towards subsequent cellular division. Cellular defects in the pathways providing effective proliferation-inhibitory mechanisms may result in accumulation of damage to DNA if sufficient time is not given for their resolution. Accumulated DNA damage may lead to irreparable lesions in the DNA which may result in cell death.

Replication of DNA, occurring predominantly in the S phase, involves faithful copying of the parental DNA, creating two identical sets double-stranded DNA chains (chromosomes). The process of replication involves assembly of a replication machinery at origins of replication which set off along the DNA strand, unravelling the double-strand DNA helix and progressively synthesizing the nascent strands in a semi-conservative fashion. The replication machinery takes in one DNA double helix on one end and creates two at the other end, creating a Y-shaped structure, termed a “replication fork”.

DNA synthesis requires an existing pool of deoxynucleotide-triphosphates (dTTP, dATP, dCTP, dGTP) to be available as building blocks for the process. The absence of any one of the nucleotide triphosphates will result in stalled replication and arrested replication forks (ref. 5). Over time, arrested replication forks can collapse, resulting in an open-ended double stranded DNA break (DSB) without a partner, a complex lesion which can only be repaired via homologous recombination-mediated replication fork re-assembly (ref. 6). The recognition of such collapsed forks is a key early event in their repair, which is orchestrated via cascade of phosphorylation events initiated through activation the Ataxia Telangiectasia-Related (ATR) kinase (ref. 7). In the absence of the repair of the above lesions, cells may undergo cell death via different mechanisms.

Inhibition of DNA synthesis is one of the ways to prevent excessive proliferation capacity of hyperplastic disorders, as well as malignant lesions (ref. 8). Many different small molecule drugs have been used to treat such lesions, however 5-fluorouracil (5-FU) has been established over the years a standard of care treatment for topical application (ref. 9). Among other activities, 5-FU works as an inhibitor of thymidylate synthase, a key enzyme in production of dTTP (ref. 10). In the presence of limiting amounts of dTTP, proliferating cells slow down DNA replication and replication forks stall. These in turn result in more frequent collapsed replication forks, which can lead to predominant killing of proliferating cells. The increased cell death upon 5-FU treatment is a balance between increased levels of collapsed replication forks (i.e. double strand DNA breaks or DSBs), activated DNA damage signalling, the remaining dTTP available to restart DNA synthesis and the ability of the cells to recognise and repair the resultant damage. The present inventor hypothesised that tipping the balance towards increasing the DSB load, and reduced repair, should increase effectiveness of 5-flurouracil and its ability to clear disorders which are only partially responsive to 5-FU treatment alone.

Therefore, there is an unmet need for an improved topical treatment of skin and/or mucous membrane lesions.

### SUMMARY OF THE INVENTION

To increase effectiveness of 5-flurouracil the inventor set out to reduce the ability of cells to repair 5-FU-induced DSBs and collapsed replication forks. The inventor hypothesised that one way to achieve this might be to prevent cellular recognition of these lesions via activation of Ataxia Telangiectasia Mutated (ATM) and Ataxia Telangiectasia Related (ATR) kinases, and if possible, inhibition of necrotic cell death through PARP inhibition as well. A class of molecules exhibition some of the properties to various extents are methylxanthines, as hypothesized previously to enhance 5FU activity (15). However, the biophysical properties of one of the methylxanitine molecules—theobromine—including limited solubility, safety, pharmacokinetics and half-life when administered to humans, as well as triple inhibitory activity (against ATM, ATR and PARP) under physiologically and clinically achievable conditions (refs. 11-14), led the inventor to hypothesize that theobromine would be the molecule with the best synergistic effect in a combination treatment with 5FU, significantly better that the best studied representative methylxantine molecule, caffeine or another clinically relevant methylxantine, theophylline.

One consequence of 5-FU treatment is both apoptotic and necrotic cell death, which cause local inflammation and discomfort during topical treatment. The inventor have discovered that theobromine works in local application to increase efficacy of 5-FU to treat skin lesions and/or reduce 5-FU mediated inflammation and pain, thus improving treatment regimen by decreasing unwanted side-effects or, in the alternative, increasing the concentration, and/or the frequency of application, of 5-FU that can be applied to the skin of a subject without causing intolerable inflammation as a side-effect. In addition, lower pain and discomfort levels may be expected to improve patient adherence to a treatment regimen.

Accordingly, in a first aspect, the invention provides a first composition comprising 5-fluorouracil (5-FU) and a second composition comprising a triple inhibitor, inhibitor of Ataxia Telangiectasia Mutated (ATM) kinase, Ataxia Telangiectasia Related (ATR) kinase and Poly-ADP-ribose polymerase (PARP) inhibitor (henceforth the triple inhibitor) for treatment of disorders in which there is hyperproliferation of keratinocytes in skin and/or mucous membranes. A preferred triple inhibitor is a methylxanthine, more specifically theobromine. In some embodiments, the second composition may comprise two or more ATM or ATR kinase inhibitors, one of which may be a methylxanthine, preferably theobromine.

The compositions may be applied topically to the skin and/or mucous membrane of a mammalian subject. The subject will advantageously be a human, usually an adult human, and preferably an adult of 50 years of age or older. Mammalian subjects other than humans may be, for instance, companion animals or farming livestock.

The compositions will preferably be formulated for topical application, using formulation methodologies and non-active ingredients which are conventional in the art. Thus, for example, the compositions may be formulated as a gel, cream, ointment, lotion, solution, suspension or the like and, in addition to the active agents, may otherwise typically comprise conventional constituents such as water, emulsifiers (e.g. polysorbate compounds, especially polysorbate 20—polysorbate 80), preservatives and antimicrobials (e.g. parabens, such as methyl hydroxybenzoate, propyl hydroxybenzoate), a dermatologically acceptable carrier, diluent or excipient, (e.g. propylene glycol, lanolin), and thickeners and emollients (e.g. stearyl alcohol). Appropriate amounts and relative proportions of these substances will be apparent to those skilled in the art and/or can be readily determined by trial and error.

The compositions may be applied separately to the affected area of the body. The sequence of application is not important. Alternatively, both compositions may be applied essentially simultaneously (i.e. the second composition is applied within 10 minutes of applying the first composition). Advantageously the compositions will be both applied to the affected part of the skin within 24 hours of one another. Typically the two respective compositions will be applied to the affected area of the body on the same day, although in some embodiments it is envisaged that the first composition may be applied on one day, the second composition applied the next day, then the first composition applied the following day, and so on, until the course of treatment has been completed. In some preferred embodiments, 5-FU is applied daily or every other day; and a triple inhibitor (preferably theobromine) is applied every day.

In a second aspect, the invention provides a single composition comprising both 5-fluorouracil and a triple inhibitor, preferably theobromine. In some embodiments, the second composition may comprise two or more inhibitors recapitulating a triple inhibitor functionality, one or more of which may be a methylxanthine, preferably theobromine.

The composition of the second aspect of the invention may preferably be for treatment of hyperproliferative disorders of keratinocytes in skin and/or mucous membranes in a mammalian, preferably human, subject. The features of the first aspect of the invention defined above will, unless the context dictates otherwise, be equally applicable to the composition of the second aspect.

The composition/s of the first or second aspects of the invention may comprise 5-fluorouracil and a triple inhibitor, preferably theobromine, 5-fluorouracil or a triple inhibitor, preferably theobromine, but may additionally also comprise one or more other substances known to have activity against hyperplastic keratinocyte disorders, such as actinic keratosis, when topically applied. For example, other substances known to have activity against actinic keratosis include: diclofenac, imiquimod (e.g. at 3-5% v/w), salicylic acid (e.g. at up to 10% v/w), ingenol mebutate (e.g. at 0.015-0.05 % v/w), and tirbanibulin (e.g. at 1% v/w).

The composition/s will comprise 5-FU and/or a triple inhibitor, preferably theobromine, at an effective concentration, that is, a concentration which, when the composition is applied to the patient, brings about a measurable improvement in the condition after a suitable period of treatment (typically this will involve daily topical application, for at least 2 days, preferably at least 1 week, more preferably at least 2 weeks, and most preferably at least three weeks). Improvement in the condition being treated can be determined, for example, by measuring the area of the visible lesion or lesions, or by counting the number of visible lesions in the treated area of skin.

Typically an effective concentration of 5-FU will be in the range 0.01 to 50% w/w or w/v, preferably in the range 0.1 to 20%, and more preferably in the range 1 to 10%, and most preferably around 5-10%.

An effective concentration of a triple inhibitor, preferably theobromine, will range from 0.01 to 50% w/w or w/v, preferably in the range 0.1 to 20%, and most preferably in the range 0.5-5%.

In a third aspect, the invention provides a method of making a composition in accordance with the second aspect of the invention, the method comprising the step of: causing to be present, in a single composition, an effective concentration of 5-FU and an effective concentration of a triple inhibitor, preferably theobromine.

Generally, the method of making a composition as defined above will further comprise mixing 5-FU and a triple inhibitor, preferably theobromine, with a dermatologically acceptable carrier, diluent or excipient, and packaging the composition in a container, such as a tube, bottle, tub or the like.

In a fourth aspect, the invention provides a method of treating hyperproliferative disorders of keratinocytes in skin and/or mucous membranes of a mammalian, preferably human, subject, the method comprising the step of applying to the skin and/or mucous membrane, as appropriate, of a patient in need of such treatment, an effective amount of first and second compositions in accordance with the first aspect of the invention, or an effective amount of a single composition in accordance with the second aspect of the invention, as defined above.

Agents contained within the composition are safe for topical application so the treatment may be applied by the patient themselves. Typically the composition is applied to the affected area of skin and/or mucous membrane on a daily basis, or possibly every other day, and the treatment is generally continued for a period of at least two weeks up to about 2-3 months. At the end of this period the patient may be assessed by a clinician (or veterinarian, as appropriate) if desired, and treatment either continued or halted, as appropriate, according to the opinion of the clinician or veterinarian.

Advantages of the various aspects of the present invention include: more effective treatment than existing therapies, and/or less inflammation, and/or less painful and inconvenient for patients. As a result of the lower levels of inflammation and/or discomfort experienced by patients with treatments/compositions in accordance with the present invention, there is improved patient compliance with prescribed or approved treatment regimens, leading to better clinical results. Alternatively, or additionally, the lower levels of inflammation and/or discomfort experienced by patients permit more frequent application of active agents, especially 5-FU, and/or topical application of the active agent (especially 5-FU) at a higher concentration, than hitherto.

In particular, the inventor have found that the combination of 5-FU and a triple inhibitor, preferably theobromine, confers synergistic results, greater than the additive effects of the two treatments in isolation.

The invention will now be further described by way of illustrative examples and with reference to the accompanying drawings in which:

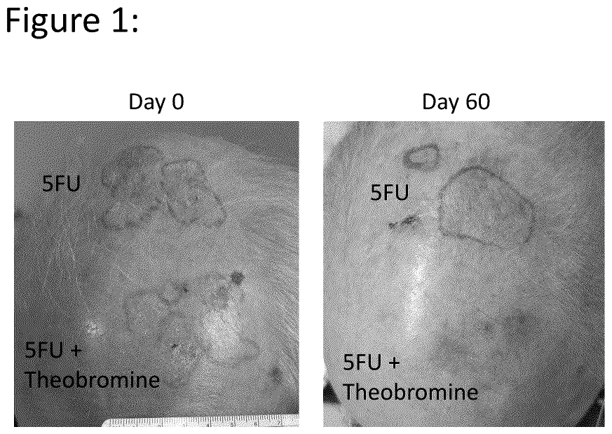

FIG. 1 shows images of actinic keratosis regions treated with either Efudix® 5-fluorouracil cream alone, or a combination of Efudix® 5-fluorouracil and 0.5% theobromine either at the onset of treatment or after 30 days of treatment followed by 30 days of recovery from treatment i.e. 60 days total, as described in Example 1 below;

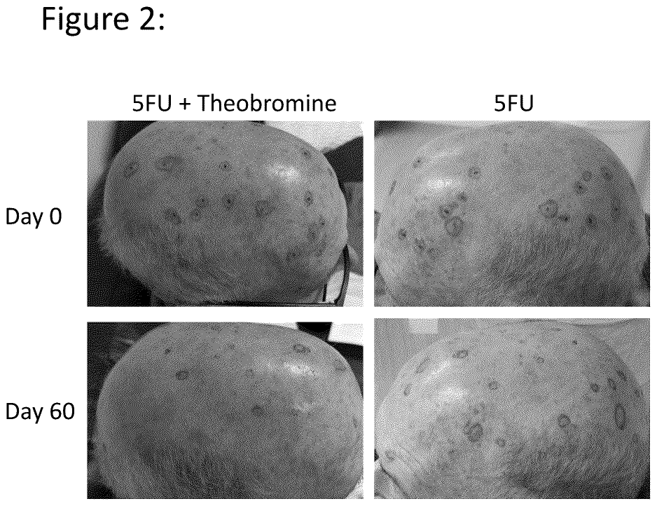

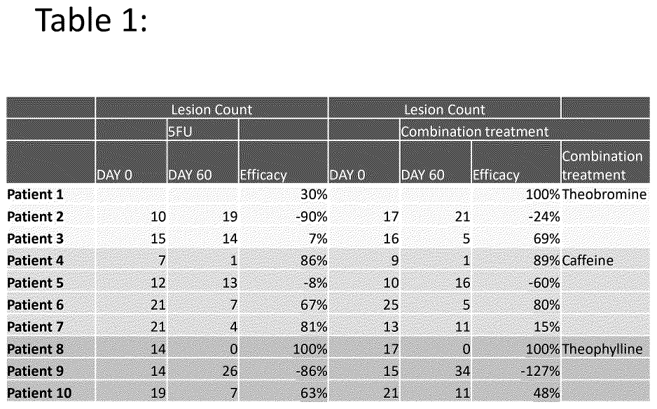

FIG. 2 shows representative images of treated skin regions on the scalp of a subject at indicated times (from day 0 to day 60 after initiation of treatment, day 60 being 30 days of treatment followed by 30 days of recovery). Medially divided scalp treatment shows images pre and post treatment on left (Efudix® alone) and right (Efudix® and 0.5% theobromine) of the scalp. Quantification is as described for Patient #2 in Table 1;

Table 1 shows quantification of individual simple AK lesions in 10 patients treated as described, and total efficacy, quantified either as reduction of AK count or affected area, patients 3-10 are as described in Example 3;

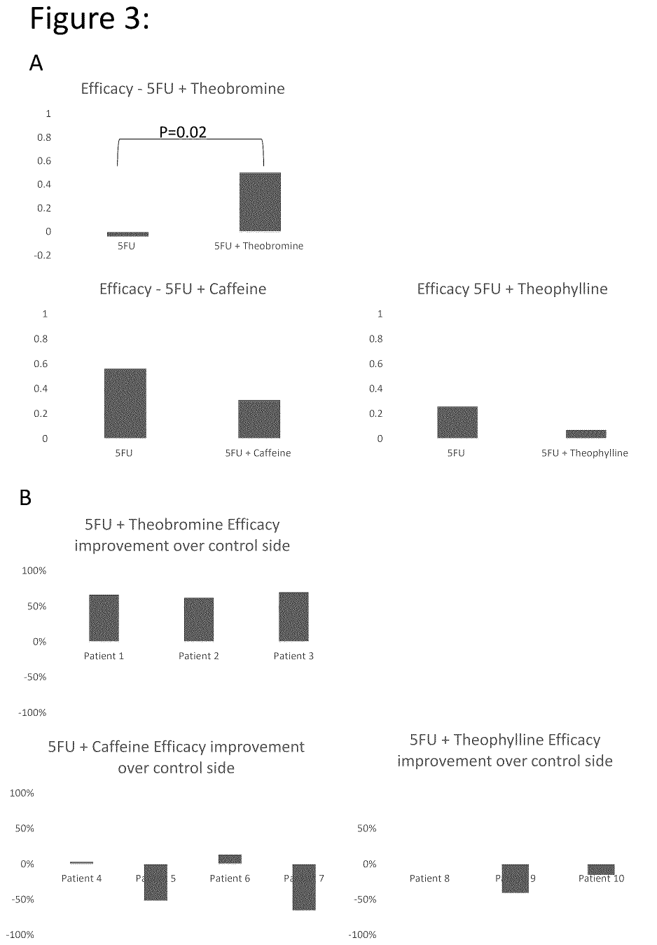

FIG. 3A Describes quantification of efficacies of treatment with either theobromine, caffeine or theophylline in combination with Efudix® 5% 5FU treatment, in paired comparison to the control region on the corresponding patients. P values below 0.05, denoting significance, are as marked.

FIG. 3B depicts changes in efficacy upon theobromine, caffeine or theophylline treatment in combination with Efudix® 5% 5FU, as compared to 5FU alone treatment on the corresponding patient as baseline, as described in the Example 3 below. Results are shown per individual patient.

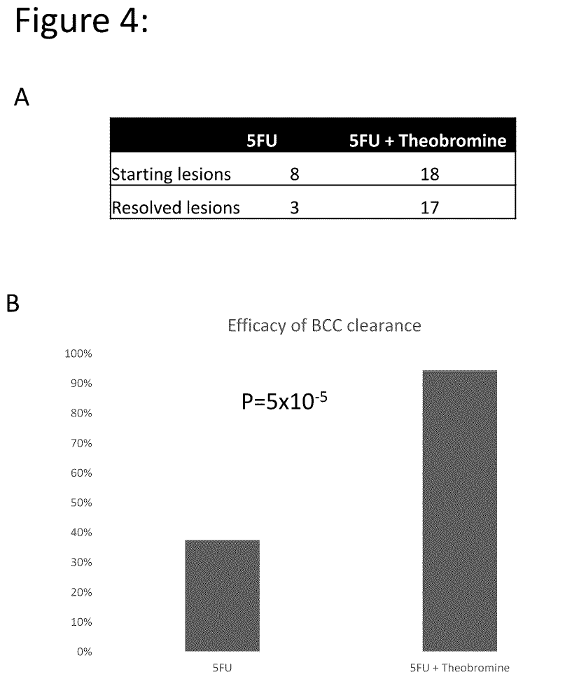

FIG. 4A Depicts number of treated lesions and treatment results as described in the Example 6 below. The initial lesions have been clinically identified by a qualified physician and the final evaluation after treatment have been performed either clinically by a qualified physician or by histopathological evaluation.

FIG. 4B is a graphical representation of the BCC clearance rate data presented in FIG. 4A. The P-value is calculated based on binomial distribution analysis.

### EXAMPLE 1

In one experiment a 81-year old man with an actinic keratoses region on his scalp was treated with 5% 5-fluorouracil cream once daily for 30 days (as marked in FIG. 1, posterior region). Another area of actinic keratoses on his scalp (As marked on FIG. 1, anterior region) was treated with both 5% 5-fluorouracil cream once daily and 0.5% theobromine solution once daily for 30 days. The 5% 5-FU cream applied was a medically available cream from Mylan, commercially known as Efudix® cream. The theobromine solution was prepared in 25% ethanol and 50% glycerol, adjusted to pH 8 via 100 mM sodium hydrogen carbonate solution. Efudix® was stored as per manufacturer's instructions, at room temperature, and theobromine solution at room temperature in the dark. In application, 50-100 μL (1-2 drops) of theobromine solution was applied first in a thin water-based layer over the whole treated area evenly. 5-FU was applied 10-20 minutes later, via finger application, as per best practice and manufacturer's instructions. One fingertip unit (~0.5 g) was used for an equivalent of half the forehead area.

After 30 days of treatment both areas treated with 5-FU alone, or 5-FU and theobromine in combination, became inflamed but actinic keratoses treated with the tested combination were less red, less sore and less inflamed. Redness and inflammation were assessed by the responsible physician. The patient reported significant discomfort with treatment: pain and soreness were reported by the patient to be 9/10 in areas treated with 5-FU alone, but the pain levels were only 7/10 in areas treated with the combination treatment.

A week following the cessation of treatment, the areas treated with 5-FU alone exhibited significant inflammation and scaly appearance, while the areas treated with the combination treatment showed reduced inflammation and scaliness in comparison.

Four weeks after the completion of treatment there was no visible residual inflammatory reaction to the treatment, no inflammation or scaliness in the areas treated with 5-FU or a combination of 5-FU and theobromine, and no pain reported by the patient. Examination showed complete clearance of the actinic keratosis lesion treated with the combination of 5-FU and theobromine. The 5-FU alone treatment resulted in incomplete clearance with the reduction in size measured to be just over 30%, as assessed using ImageJ image analysis software.

The results of the experiment demonstrate that not only was the combination treatment of 5FUand theobromine more effective than 5FU alone in clearing actinic keratosis lesions, but also that the combination treatment caused very substantially less pain, discomfort and inflammation during, and immediately after, treatment than 5FU alone.

### EXAMPLE 2

In another experiment a 70-year old man with an actinic keratoses on his scalp was treated with 5% 5-fluorouracil cream 4× per week for 4 weeks (15 lesions, as marked in FIG. 2, left side of scalp). Another area of actinic keratoses on his scalp (16 lesions, as marked on FIG. 2, right side of scalp) was treated with both 5% 5-fluorouracil cream 4× per week and 0.5% theobromine solution 4× per week for 4 weeks. The 5% 5-FU cream applied was a medically available cream from Mylan, commercially known as Efudix® cream. The theobromine solution was prepared in 25% ethanol and 50% glycerol, adjusted to pH 8 via 100 mM sodium hydrogen carbonate solution. Efudix® was stored as per manufacturer's instructions, at room temperature, and theobromine solution at room temperature in the dark. In application, 50-100 μL (1-2 drops) of theobromine solution was applied first in a thin water-based layer over the whole treated area evenly. 5-FU was applied 10-20 minutes later, via finger application, as per best practice and manufacturer's instructions. One fingertip unit (~0.5 g) was used for an equivalent of half the forehead area.

After 4 weeks of treatment both areas treated with 5-FU alone, or 5-FU and theobromine in combination, became inflamed but actinic keratoses treated with the tested combination were less red, less sore and less inflamed. Redness and inflammation were assessed by the responsible physician. The patient reported mild discomfort with treatment: pain and soreness were reported by the patient to be 2/10 in areas treated with 5-FU alone, and the pain levels were 1/10 in areas treated with the combination treatment.

Four weeks after the completion of treatment there was no visible residual inflammatory reaction to the treatment, no inflammation or scaliness in the areas treated with 5-FU or a combination of 5-FU and theobromine, and no pain reported by the patient. Examination showed improved clearance of the actinic keratosis lesions treated with the combination of 5-FU and theobromine, with only 5 lesions identifiable by the qualified physician. The 5-FU alone treatment also resulted in incomplete clearance, however the reduction in lesion count was minimal, with lesion count reduced from starting 15 to 14.

The results of the experiment again demonstrate that not only was the combination treatment of 5FU and theobromine more effective than 5FU alone in clearing actinic keratosis lesions, but also that the combination treatment caused less pain, discomfort and inflammation during, and immediately after, treatment than 5FU alone.

### EXAMPLE 3

Following above results, inventor tested whether the ability to positively affect 5FU treatment was limited to theobromine alone. To this end, caffeine and theophylline solution were prepared at 0.5% w/v in the same way as theobromine in Examples 1 and 2. 8 more patients ranging in age from 52 to 81 were treated in an analogous way as in Example 2, with one patient receiving treatment with 5FU +0.5% w/v theobromine, four patients with caffeine 0.5% w/v and 3 patients with theophylline 0.5% w/v. All patients were treated solely on the scalp and were only treated on half of the scalp with the test compound and 5FU, while the other half was used as control, treated with 5FU alone. Separation between combination and 5FU only control was mostly done medially, with one patient having and anterior/posterior treatment setup. As in Example 2, the patients self-administered the test compound on the test region 10-20 minutes before applying the Efudix® cream containing 5% w/v 5FU. In application, 50-100 μL (1-2 drops) of the test compound solution was applied first in a thin water-based layer over the whole treated area evenly. 5-FU was applied 10-20 minutes later, via finger application, as per best practice and manufacturer's instructions. Patients applied the regimen at regular intervals, once daily. One fingertip unit (~0.5 g) was used for an equivalent of half the scalp area.

The results were significantly varied (See Table 2), which the inventor ascribe to a combination of underlying genetics and the effect of previous treatments for AK that many of the patients have had before. Nevertheless, there are key differences in theobromine activity versus other methylxantines.

When statistically analysed, notwithstanding the variability in the sample set, theobromine treatment significantly improved AK lesion clearance on patients, with improved efficacy over control that was statistically significant (p=0.003). Treatment with either caffeine or theophylline resulted in decreased efficacy, however the result did not reach statistical significance (FIG. 3A)

Due to variability between patients, it is advisable to also assess trends in patients, to see if particular treatments indicate particular functionalities on a per patient basis. Notably, in this case again, only combination of 5FU and theobromine showed a consistent improvement in efficacy of about 60% over expected efficacy, while neither caffeine not theophylline showed any positive trends (FIG. 3B).

Together, this indicated theobromine as a novel compound that can enhance efficacy of 5FU treatment, uniquely capable of doing so when compared to other methylxanthines readily available for clinical applications at identical concentrations.

### EXAMPLE 4

In another example, a 60-year old woman with multiple basal cell carcinomas was treated with: 5% 5-fluorouracil (5-FU) cream alone (1 lesion, back, clinical diagnosis) or a combination of 5-FU cream and theobromine solution (2 lesions-biopsy confirmed basal cell carcinoma, right leg and basal cell carcinoma, left forearm, clinical diagnosis). The treatment was continued once daily for 1 month and 0.5% percent theobromine solution. In application, 50-100 μL (1-2 drops) of theobromine solution was applied in a thin water-based layer over the whole treated area evenly (up to 50 cm2 area in total). 5-FU was applied via finger application, as per best practice and manufacturer's instructions. One finger tip unit (0.5 g) was used to cover 1 lesion. 5FU was applied 30 min after theobromine. One month after completing treatment the patient was examined by a dermatologist. The lesions treated with 5FU and theobromine resolved and the lesion treated with 5FU persisted.

### EXAMPLE 5

In another example a 81-year old woman with a clinical diagnosis of basal cell carcinoma was treated with Efudix® cream containing 5% w/v 5FU and 0.5% caffeine solution. In application, 50-100 μL (1-2 drops) of caffeine solution was applied in a thin water-based layer over the whole treated area evenly (up to 50 cm2 area in total). 5-FU was applied via finger application, as per best practice and manufacturer's instructions. One finger tip unit (0.5 g) was used to cover the lesion. At the end of the treatment she was examined by a dermatologist. The treatment was continued once daily for 1 month. Upon medical examination, it was observed that the bcc persisted.

### EXAMPLE 6

In another example 6 patients (age 50 to 86) with one or more basal cell carcinomas were treated as in the example 4 with either 5% 5-fluorouracil (5-FU) cream alone or with a combination of 5-FU cream and 0.5% percent theobromine solution. In application, 50-100 μL (1-2 drops) of theobromine solution was applied in a thin water-based layer over the whole treated area evenly (up to 50 cm2 area in total). 5-FU was applied via finger application, as per best practice and manufacturer's instructions. One finger tip unit (0.5 g) was used to cover 1 lesion. 5FU was applied 30 min after theobromine. One month after completing treatment the patients was examined by a dermatologist. Overall 8 basal cell carcinomas were treated with 5-fluorouracil and 18 basal cell carcinomas were treated with a combination of 5-FU cream and theobromine solution. The results of treatments are depicted in FIG. 4. The 5-FU cream and theobromine combination treatment resulted in a better efficacy of treatment of basal cell carcinomas comparing to the treatment with 5-fluorouracil alone.

### EXAMPLE 7 (COMPARATIVE EXAMPLE, NOT IN ACCORDANCE WITH THE INVENTION)

In another example a 60-year old woman with a clinical diagnosis of basal cell carcinoma was treated with 0.5% percent theobromine solution. In application, 50-100 μL (1-2 drops) of theobromine solution was applied in a thin water-based layer over the whole treated area evenly (up to 50 cm 2 area in total). Theobromine was applied once daily for 2 months. At the end of the treatment, she was examined by a dermatologist. Upon medical examination, it was observed that the basal cell carcinoma persisted. No evidence of inflammation or pain and no change in the lesion appearance was observed.

### EXAMPLE 8 (COMPARATIVE EXAMPLE, NOT IN ACCORDANCE WITH THE INVENTION)

In another example a 61-year old man with a clinical diagnosis of basal cell carcinoma was treated with 0.5% percent theobromine solution. In application, 50-100 μL (1-2 drops) of theobromine solution was applied in a thin water-based layer over the whole treated area evenly (up to 50 cm 2 area in total). Theobromine was applied once daily for 1 months. The patient was examined by a dermatologist before the treatment and one week after completion of the treatment. Upon medical examination, it was observed that the basal cell carcinoma persisted. No evidence of inflammation or pain and no change in the lesion size or appearance was observed.

### EXAMPLE 9: (COMPARATIVE EXAMPLE, NOT IN ACCORDANCE WITH THE INVENTION)

In another experiment a 70-year old man with an area of actinic keratoses on his scalp was treated with 0.5% theobromine solution once daily for 4 weeks. The theobromine solution was prepared in 25% ethanol and 50% glycerol, adjusted to pH 8 via 100 mM sodium hydrogen carbonate solution. Theobromine solution was stored at room temperature in the dark. In application, 50-100 μL (1-2 drops) of theobromine solution was applied first in a thin water-based layer over the whole treated area evenly.

After 4 weeks of treatment the area did not became inflamed with no redness or inflammation detected by the responsible physician or the patient. The patient reported no discomfort with treatment: pain and soreness were reported by the patient to be 0/10 in areas treated with theobromine treatment. No reduction in a number of actinic keratoses was observed by a responsible physician. The results of the experiment demonstrate that theobromine alone does not reduce number of actinic keratoses.

### EXAMPLE 10: (COMPARATIVE EXAMPLE, NOT IN ACCORDANCE WITH THE INVENTION)

In another experiment a 81 year old man with an area of actinic keratoses on his nose was treated with 0.5% theobromine solution once daily for 4 weeks. The theobromine solution was prepared in 25% ethanol and 50% glycerol, adjusted to pH 8 via 100 mM sodium hydrogen carbonate solution. Theobromine solution was stored at room temperature in the dark. In application, 50-100 μL (1-2 drops) of theobromine solution was applied first in a thin water-based layer over the whole treated area evenly.

After 4 weeks of treatment the area did not became inflamed with no redness or inflammation detected by the responsible physician or the patient. The patient reported no discomfort with treatment: pain and soreness were reported by the patient to be 0/10 in areas treated with theobromine treatment. No reduction in a number of actinic keratoses was observed by a responsible physician 1 week after completion of treatment. The results of the experiment demonstrate that theobromine alone does not treat actinic keratoses.

Examples of formulations of compositions in accordance with the invention:

### EXAMPLE 11

In a specific embodiment, a composition in accordance with the second aspect of the present invention comprises: (a) 5-fluorouracil, present in the composition at a concentration of 5% w/w and (b) theobromine, present in the composition at a concentration of 0.5 % w/w, (a) and (b) being the active ingredients. In addition, the composition contains:
(c) olive oil (20% w/w);
(d) lanolin (6% w/w);
(e) glycerine 5% w/w);
(f) propylene glycol (20% w/w);
(g) polyethylene glycol 400 (23% w/w);
(h) isopropyl myristate (3% w/w); and
(i) an aqueous buffer solution adjusted to an appropriate pH (e.g. in the range 6.5-7.5).

Components of the formulation are filter sterilised by passage through a 0.22 mm pore filter as necessary and the formulation is stored at 4 °C until required.

The skin and/or mucous membrane, as appropriate, is treated with above topical preparation. The cream is topically applied to the entire area to be treated, at ambient temperature, about 18-20 °C. The formulation is left on the subject's skin for 60 minutes and then if desired the area can be wiped or washed. The treatment is repeated 3 times a week for 1 month.

### EXAMPLE 12

In a specific embodiment, a composition in accordance with the second aspect of the present invention comprises: (a) 5-fluorouracil, present in the composition at a concentration of 2% w/w; and (b) theobromine, present in the composition at a concentration of 0.5% w/w, with (a) and (b) being the active ingredients. In addition, the composition contains:
(c) isopropyl myristate (25% w/w);
(d) polyoxyethylene (5% w/w);
(e) cetyl ether (5% w/w);
(f) carbopol 940 (1% w/w); and
(g) an aqueous buffer solution adjusted to an appropriate pH (e.g. in the range 6.5-7.5).

Components of the formulation are filter sterilised by passage through a 0.22 mm pore filter as necessary and the formulation is stored at 4 °C until required.

The skin and/or mucous membrane, as appropriate, is treated with above topical preparation. The cream is topically applied to the entire area to be treated, at ambient temperature, about 18-20 °C. The formulation is left on the subject's skin for 60 minutes and then if desired the area can be wiped or washed. The treatment is repeated once daily for 1 month.

### REFERENCES

1. Uhlenhake EE. Optimal treatment of actinic keratoses. Clin Interv Aging. 2013; 8:29-35.
2. Balcere A, Rone Kupfere M, Čēma I, Krūmiņa A. Prevalence, Discontinuation Rate, and Risk Factors for Severe Local Site Reactions with Topical Field Treatment Options for Actinic Keratosis of the Face and Scalp. Medicina (Kaunas). 2019 April 4; 55(4):92.
3. Chen O M, Kim K, Steele C, et al Advances in Management and Therapeutics of Cutaneous Basal Cell Carcinoma. Cancers (Basel). 2022; 14(15):3720
4. Tanese K. Diagnosis and Management of Basal Cell Carcinoma. Curr Treat Options Oncol. 2019 February 11; 20(2):13.
5. Gelot C, Magdalou I, Lopez BS. Replication stress in Mammalian cells and its consequences for mitosis. Genes (Basel). 2015; 6(2):267-298.
6. Cortez D. Preventing replication fork collapse to maintain genome integrity. DNA Repair (Amst). 2015; 32:149-157. doi: 10.1016/j. dnarep.2015.04.026
7. Saldivar J C, Cortez D, Cimprich K A. The essential kinase ATR: ensuring faithful duplication of a challenging genome [published correction appears in Nat Rev Mol Cell Biol. 2017 Dec.; 18(12):783]. Nat Rev Mol Cell Biol. 2017; 18(10):622-636. doi: 10.1038/nrm.2017.67
8. Ubhi T, Brown G W. Exploiting DNA Replication Stress for Cancer Treatment. Cancer Res Apr. 15 2019(79) (8) 1730-1739; DOI: 10.1158/0008-5472.CAN-18-3631
9.https://www.bad.org.uk/patient-information-leaflets/5-fluorouracil-cream/?showmore=1&returnlink=https%3a%2f%2fwww.bad.org.uk%2fpatient-information-leaflets#.YQAxFOhKiMo
10. Longley D B, Harkin D P, Johnston P G. 5-fluorouracil: mechanisms of action and clinical strategies. Nat Rev Cancer. 2003 May; 3(5):330-8. doi: 10.1038/nrc1074. PMID: 12724731.
11. Hall-Jackson, C., Cross, D., Morrice, N. et al. ATR is a caffeine-sensitive, DNA-activated protein kinase with a substrate specificity distinct from DNA-PK. Oncogene 18, 6707-6713 (1999). https://doi.org/10.1038/sj.onc.1203077
12. Musk S R, Steel G G. Override of the radiation-induced mitotic block in human tumour cells by methylxanthines and its relationship to the potentiation of cytotoxicity. Int J Radiat Biol. 1990 June; 57(6): 1105-12
13. Geraets L, Moonen H J, Wouters E F, Bast A, Hageman G J. Caffeine metabolites are inhibitors of the nuclear enzyme poly(ADP-ribose)polymerase-1 at physiological concentrations. Biochem Pharmacol. 2006 September 28; 72(7):902-10
14. Proceedings of the 103rd Annual Meeting of the American Association for Cancer Research; 2012 Mar. 31-Apr 4; Chicago, IL. Philadelphia (PA): AACR; Cancer Res 2012; 72(8 Suppl): Abstract nr 5716
15. Lu Y, Conney AH, Lou Y, Caffeine salt complexes and methods for using the same I the prevention or treatment of Cancer, (2005), US020050207998A1

1. A first composition comprising 5-fluorouracil, and a second composition comprising a triple (Ataxia Telangiectasia Mutated (ATM) kinase, Ataxia Telangiectasia Related (ATR) kinase, Poly-ADP ribose polymerase (PARP)) inhibitor, preferably theobromine, for the use in the treatment of an actinic keratosis of the skin and/or mucous membrane of a mammalian, preferably human, subject.

2. A first composition comprising 5-fluorouracil, and a second composition comprising a triple (Ataxia Telangiectasia Mutated (ATM) kinase, Ataxia Telangiectasia Related (ATR) kinase, Poly-ADP ribose polymerase (PARP)) inhibitor, preferably theobromine, for the use in the treatment of a basal cell carcinoma of the skin and/or mucous membrane of a mammalian, preferably human, subject.

3. A composition comprising 5-fluorouracil and a triple (Ataxia Telangiectasia Mutated (ATM) kinase, Ataxia Telangiectasia Related (ATR) kinase, Poly-ADP ribose polymerase (PARP)) inhibitor, preferably theobromine.

4. A composition according to claim 1, or formulated for topical application to the skin and/or mucous membrane of a mammalian, preferably human, subject.

5. A composition according to claim 1, wherein the inhibitor of ATM kinase, ATR kinase and PARP inhibitor is theobromine.

6. A composition according claim 1 further comprising one or more of the substances selected from the group consisting of:
emulsifiers; preservatives and antimicrobials; solvents, carriers or diluents; and thickeners and emollients.

7. A composition according to claim 6, comprising one or more of: water; parabens; polysorbates; and glycerol.

8. A composition according to claim 1, further comprising a substance having known activity against activity actinic keratosis, or basal cell carcinoma.

9. A composition according to claim 8, comprising one or more substances selected from the group consisting of: diclofenac; imiquimod; salicylic acid; ingenol mebutate; tirbanibulin; and trichloracetic acid, imiquimod.

10. A composition according to claim 1, comprising 5-fluorouracil at a concentration in the range 0.01 to 50% w/w or w/v, preferably in the range 0.1 to 20%, and more preferably in the range 1 to 10%.

11. A composition according to claim 1, comprising a triple (Ataxia Telangiectasia Mutated (ATM) kinase, Ataxia Telangiectasia Related (ATR) kinase, Poly-ADP ribose polymerase (PARP)) inhibitor at a concentration in the range 0.001 to 50% w/w or w/v, preferably in the range 0.01 to 10%, and more preferably in the range 0.1 to 2%, and further wherein the inhibitor is preferably theobromine.

12. A method of making a composition in accordance with claim 3, the method comprising the step of: causing to be present, in a single composition, an effective concentration of 5-FU and an effective concentration of a triple (Ataxia Telangiectasia Mutated (ATM) kinase, Ataxia Telangiectasia Related (ATR) kinase, Poly-ADP ribose polymerase (PARP)) inhibitor preferably theobromine.

13. A method according to claim 12, further comprising the step of mixing 5-FU and an inhibitor of a triple (Ataxia Telangiectasia Mutated (ATM) kinase, Ataxia Telangiectasia Related (ATR) kinase, Poly-ADP ribose polymerase (PARP)) inhibitor with a dermatologically acceptable carrier, diluent or excipient, and packaging the composition in a container.

14. A method according to claim 12, wherein the inhibitor of triple (Ataxia Telangiectasia Mutated (ATM) kinase, Ataxia Telangiectasia Related (ATR) kinase, Poly-ADP ribose polymerase (PARP)) is theobromine.

15. A method of treating a actinic keratoses or basal cell carcinoma of the skin and/or mucous membrane, the method comprising the step of: applying to the skin and/or mucous membrane of a mammalian, preferably human, subject in need of such treatment an effective amount of first and second compositions in accordance with claim 1.

16. A method according to claim 15, wherein the triple (ATM kinase, ATR kinase, PARP) inhibitor is theobromine.

17. A method according to claim 15, wherein the disorder to be treated is selected from the group consisting of: actinic keratosis; basal cell carcinoma.
